(19) United States
(12) Patent Application Publication
(10) Pub. No.: US 2024/0294510 A1
(43) Pub. Date: Sep. 5, 2024

(54) MESEMBRINE DERIVATIVES

(71) Applicant: Justin Kirkland, Fort Mill, SC (US)

(72) Inventor: Justin Kirkland, Fort Mill, SC (US)

(21) Appl. No.: 18/622,877

(22) Filed: Mar. 30, 2024

Related U.S. Application Data
(63) Continuation of application No. PCT/US2022/047047, filed on Oct. 18, 2022.
(60) Provisional application No. 63/257,453, filed on Oct. 19, 2021.

(51) Int. Cl. C07D 405/04; A61K 31/403; A61K 31/404

(52) U.S. Cl. CPC C07D 405/04; A61K 31/403; A61K 31/404

(57) ABSTRACT

Disclosed herein are 3′,4′ “methylenedioxy” analogs of the natural alkaloids and analogs of mesembrine. For example, the present disclosure provides 3′,4′-methylenedioxy-mesembrine, 3′,4′-methylenedioxy-delta-7-mesembrenone, 3′,4′-methylenedioxy-mesembrano, 3′,4′-methylenedioxy-mesembrenonel, 3′,4′-methylenedioxy-mesembrane, 3′,4′-methylenedioxy-N-demethylmesembrenol, 3′,4′-methylenedioxy-N-demethylmesembranol, 3′,4′-methylenedioxy-joubertinanmine, 3′,4′-methylenedioxy-epijoubertinanmine (chirality signified by “epi”), 3′,4′-methylenedioxy-tortuosamine, 3′,4′methylenedioxy-N-demethyl-mesembrenone, 3′,4′methylenedioxy-N-demethyl-N-formyl-mesembrenone, 3′,4′methylenedioxy-O-acetylmesembranol, 3′,4′-methylenedioxy-N-formyltortuosamine, and 3′,4′-methylenedioxy-N-acetyltortuosamine.

### CROSS-REFERENCE TO RELATED APPLICATIONS

The present application is a continuation of International Application No. PCT/US22/47047 filed on Oct. 18, 2022, which application claims the benefit of the filing date of U.S. Provisional Patent Application No. 63/257,453 filed on Oct. 19, 2021, the disclosure of which is hereby incorporated by reference herein in its entirety.

### FIELD OF THE INVENTION

Disclosed herein are 3′,4′ “methylenedioxy” analogs of the natural alkaloids and analogs of mesembrine, formulations including such analogs, and methods of treating a subject with one or more such analogs.

### BACKGROUND OF THE DISCLOSURE

Kanna is the common name of the plant Sceletium tortuosum. It is a short succulent found in South Africa. Groups of South African tribes have used the plant as a mood-altering substance from prehistoric times. The first known written account of the plant's use was in 1662 by Jan van Riebeeck. The traditionally prepared dried Sceletium was often chewed, snuffed, or smoked. It provided energy for hunter-gatherers to continue their pursuit of game or collection of food plants. There are greater than 32 bioactive alkaloids of a similar structure found in this plant. The most studied alkaloid in Kanna is mesembrine. Mesembrine exhibits activity as both a serotonin reuptake inhibitor and as a monoamine releasing agent. This activity upon the serotonin receptor has shown clinical efficacy as an antidepressant.

### BRIEF SUMMARY OF THE DISCLOSURE

A first aspect of the present disclosure is a compound having any one of any one of Formulas (VA), (VB), (VC), (VD), (VE), or (VF):

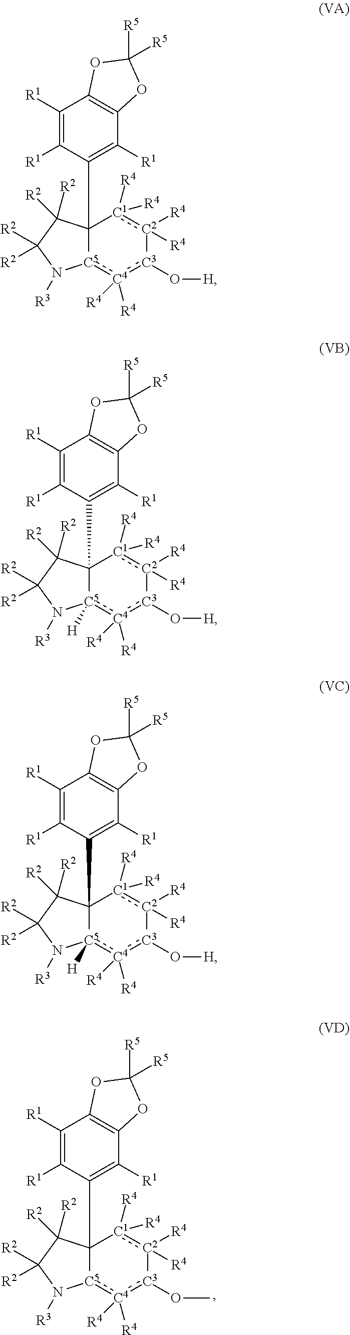

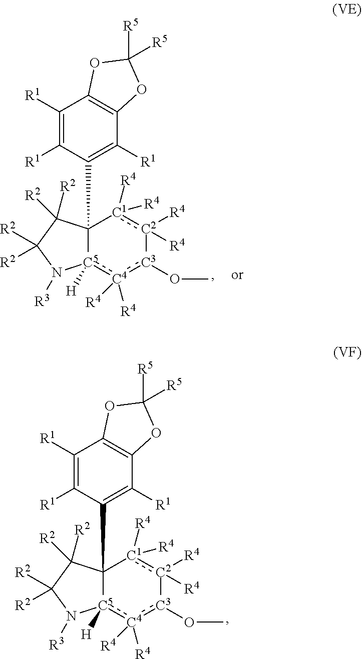

wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
A is O or S;
R⁵ is a C₁-C₄ alkyl; and
where any one of the bonds between C¹ and C², C³ and C⁴, and/or C⁴ and C⁵ may be single bonds or double bonds.

In some embodiments, at least one R is methyl. In some embodiments, is methyl. In some embodiments, each R¹ and each R² is H.

In some embodiments, each R⁵ is H. In some embodiments, R³ is methyl.

In some embodiments, each R¹, each R², and each R⁴ is H. In some embodiments, R³ is methyl.

A second aspect of the present disclosure is a compound having any one of Formulas (IVA), (IVB), or (IVC):

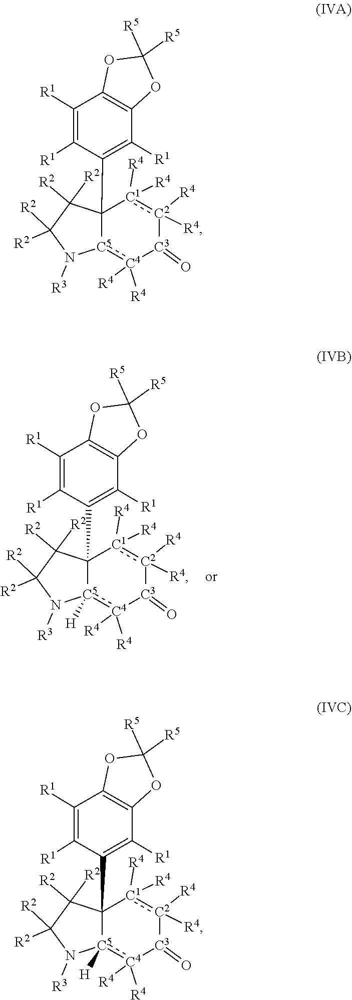

wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
A is O or S;
R⁵ is a C₁-C₄ alkyl; and
where the bonds between C¹ and C² and/or between C⁴ and C⁵ may be single bonds or double bonds.

In some embodiments, at least one R is methyl. In some embodiments, R³ is methyl. In some embodiments, each R¹ and each R² is H.

In some embodiments, each R⁵ is H. In some embodiments, R³ is methyl.

In some embodiments, each R¹, each R², and each R⁴ is H. In some embodiments, R³ is methyl.

A third aspect of the present disclosure is a compound having any one of the Formulas (VIIIA), (VIIIB), or (VIIIC):

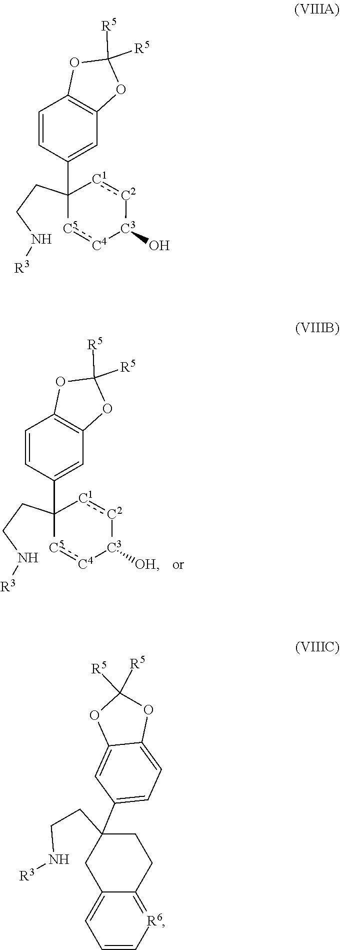

wherein
R³ is methyl or ethyl;
each R⁵ is independently H or a C₁ to C₄ alkyl group;
R⁶ is O, NH, NCH₃, or S; and
wherein the bonds between C¹ and C² and/or between C⁴ and C⁵ may be single bonds or double bonds.

In some embodiments, R⁶ is N.

In some embodiments, R³ is methyl.

In some embodiments, each R⁵ is H.

A fourth aspect of the present disclosure is a compound selected from the group consisting of:

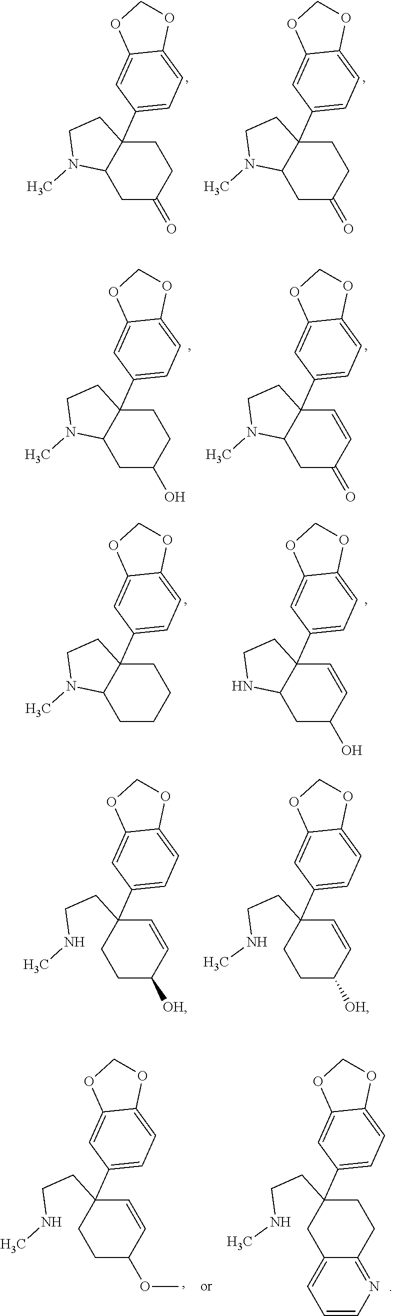

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl or aryl group, or the total number of carbon atoms and heteroatoms in a heteroalkyl, heterocyclyl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂, CH₃CH₂CH(CH₃)— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, the term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 50 or fewer carbon atoms in its backbone (e.g., C₁-C₅₀ for straight chain, C₁-C₅₀ for branched chain).

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkonyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and urcido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. An “alkylaryl” or an “arylalkyl” moiety is an alkyl substituted with an aryl (e.g., phenylmethyl (benzyl)).

As used herein, the terms “analog” or “derivative” are used in accordance with its plain ordinary meaning within chemistry and biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

Each of the terms (e.g., “alkyl,” “aromatic,” “heteroalkyl,” “cycloalkyl,” “heterocyclic,” etc.) includes both substituted and unsubstituted forms of the indicated radical. In that regard, whenever a group or moiety is described as being “substituted” or “optionally substituted” (or “optionally having” or “optionally comprising”) that group may be unsubstituted or substituted with one or more of the indicated substituents. Likewise, when a group is described as being “substituted or unsubstituted” if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an ether, amino (e.g. a mono-substituted amino group or a di-substituted amino group), and protected derivatives thereof. Any of the above groups may include one or more heteroatoms, including O, N, or S. For example, where a moiety is substituted with an alkyl group, that alkyl group may comprise a heteroatom selected from O, N, or S (e.g. —(CH₂—CH₂—O—CH₂—CH₃)).

As used herein, the term “heteroatom” is meant to include boron (B), oxygen (O), nitrogen (N), sulfur (S), phosphorus (P), and silicon (Si). As noted herein, in some embodiments, a “heterocyclic ring” may comprise one or more heteroatoms. In other embodiments, an aliphatic group may comprise or be substituted by one or more heteroatoms.

As used herein, the term “pharmaceutical composition” it is meant a composition containing an active agent as described herein, formulated with at least one pharmaceutically acceptable excipient, carrier, or diluent. The pharmaceutical composition can be manufactured or sold with the approval of a governmental regulatory agency as part of a therapeutic regimen for the treatment or prevention of a disease or event in a patient (e.g., an infant with HPP, such as an infant having perinatal-onset HPP, or an infant having infantile-onset HPP, or juvenile-onset HPP, or a patient having childhood-onset HPP). Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

As used herein, the term “subject” refers to any animal subject including laboratory animals (e.g., primates, rats, mice), livestock (e.g., cows, sheep, goats, pigs, turkeys, chickens), household pets (e.g., dogs, cats, rodents, etc.), and humans.

As used herein, the terms “treatment” or “treating” refer to any beneficial or desirable effect on the symptoms or pathology of a disease or pathological condition and may include even minimal reductions in one or more measurable markers of the disease or condition being treated. In some embodiments, the treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The present disclosure is directed to 3′,4′ methylenedioxy analogs of the natural alkaloids and analogs of mesembrine. In some embodiments, the compounds of the present disclosure have the structure of any one of Formulas (IA), (IB), or (IC):

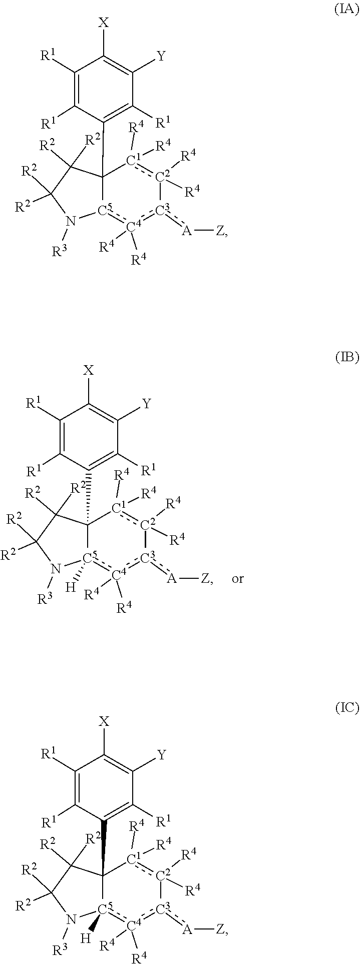

wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
X and Y are each independently —OH, —O-alkyl, —N-alkyl, —S-alkyl, or where X and Y together may form a 5- or 6-membered saturated or unsaturated cyclic or heterocyclic ring optionally substituted with one or more lower alkyl or lower alkoxy groups;
A is O or S;
Z is H, a C₁-C₄ alkyl group, or one or more pairs of electrons; and
where the bonds between C¹ and C², between C³ and C⁴, between C⁴ and C⁵, and/or between C³ and A may be single bonds or double bonds.

In some embodiments, A is O. In some embodiments, A is O and Z is H. In some embodiments, A is O and Z is methyl or ethyl. In some embodiments, A is O and Z is methyl.

In some embodiments, Z is H or methyl; and R³ is methyl. In some embodiments, Z is methyl and R³ is methyl.

In some embodiments, A is O; Z is H or methyl; and R³ is methyl. In some embodiments, A is O; Z is methyl and R³ is methyl. In some embodiments, A is O; Z is H or methyl; and R³ is ethyl. In some embodiments, A is O; Z is methyl and R³ is ethyl.

In some embodiments, A is O; the bond between C³ and A is a double bond; and R³ is methyl. In some embodiments, A is O; the bond between C³ and A is a double bond; and R³ is ethyl/

In some embodiments, X and Y together form a may form a 5- or 6-membered saturated or unsaturated cyclic or heterocyclic ring optionally substituted with one or more lower alkyl groups. In some embodiments, X and Y together form a may form a 5- or 6-membered saturated or unsaturated cyclic or heterocyclic ring optionally substituted with one or more lower alkyl groups; and where A is O. In some embodiments, X and Y together form a may form a 5- or 6-membered saturated or unsaturated cyclic or heterocyclic ring optionally substituted with one or more lower alkyl groups; where A is O; and Z is methyl. In some embodiments, X and Y together form a may form a 5- or 6-membered saturated or unsaturated cyclic or heterocyclic ring optionally substituted with one or more lower alkyl groups; where A is O; and Z is H.

In some embodiments, A is O. In some embodiments, A is O and Z is H. In some embodiments, A is O and Z is methyl or ethyl. In some embodiments, A is O and Z is methyl.

In some embodiments, one R¹ group is methyl or ethyl, while the other R¹ groups are each H. In some embodiments, one R 1 is methyl or ethyl, while the other R¹ groups are each H; and one R² group is methyl or ethyl, while the other R² groups are each H. In some embodiments, one R² is methyl or ethyl, while the other R² groups are each H; and one R⁴ group is methyl or ethyl, while the other R⁴ groups are each H. In some embodiments, one R¹ is methyl or ethyl, while the other R¹ groups are each H; and one R⁴ group is methyl or ethyl, while the other R⁴ groups are each H. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H. In some embodiments, each of the R¹, R², and R⁴ groups are H.

In some embodiments, one R¹ group is methyl or ethyl, while the other R¹ groups are each H; and wherein A is O. In some embodiments, one R 1 is methyl or ethyl, while the other R¹ groups are each H; one R² group is methyl or ethyl, while the other R² groups are each H; and wherein A is O. In some embodiments, one R² is methyl or ethyl, while the other R² groups are each H; one R⁴ group is methyl or ethyl, while the other R⁴ groups are each H; and wherein A is O. In some embodiments, one R¹ is methyl or ethyl, while the other R¹ groups are each H; one R⁴ group is methyl or ethyl, while the other R⁴ groups are each H; and wherein A is O. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and wherein A is O. In some embodiments, each of the R¹, R², and R⁴ groups are H; and A is O.

In some embodiments, one R¹ group is methyl or ethyl, while the other R¹ groups are each H; and wherein A is ═O. In some embodiments, one R 1 is methyl or ethyl, while the other R¹ groups are each H; one R² group is methyl or ethyl, while the other R² groups are each H; and wherein A is ═O. In some embodiments, one R² is methyl or ethyl, while the other R² groups are each H; one R⁴ group is methyl or ethyl, while the other R⁴ groups are each H; and wherein A is ═O. In some embodiments, one R¹ is methyl or ethyl, while the other R¹ groups are each H; one R⁴ group is methyl or ethyl, while the other R⁴ groups are each H; and wherein A is ═O. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and wherein A is ═O. In some embodiments, each of the R¹, R², and R⁴ groups are H; and A is ═O.

In some embodiments, one R¹ group is methyl or ethyl, while the other R¹ groups are each H; R³ is methyl; and wherein A is ═O. In some embodiments, one R 1 is methyl or ethyl, while the other R¹ groups are each H; one R² group is methyl or ethyl, while the other R² groups are each H; R³ is methyl; and wherein A is ═O. In some embodiments, one R² is methyl or ethyl, while the other R² groups are each H; one R⁴ group is methyl or ethyl, while the other R⁴ groups are each H; R³ is methyl; and wherein A is ═O. In some embodiments, one R¹ is methyl or ethyl, while the other R¹ groups are each H; one R⁴ group is methyl or ethyl, while the other R⁴ groups are each H R³ is methyl; and wherein A is ═O. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; R³ is methyl; and wherein A is ═O. In some embodiments, each of the R¹, R², and R⁴ groups are H; R³ is methyl; and A is ═O.

In some embodiments, one R¹ group is methyl or ethyl, while the other R¹ groups are each H; and wherein A-Z is O—Me. In some embodiments, one R¹ is methyl or ethyl, while the other R¹ groups are each H; one R² group is methyl or ethyl, while the other R² groups are each H; and wherein A-Z is O—Me. In some embodiments, one R² is methyl or ethyl, while the other R² groups are each H; one R⁴ group is methyl or ethyl, while the other R⁴ groups are each H; and wherein A-Z is O—Me. In some embodiments, one R¹ is methyl or ethyl, while the other R¹ groups are each H; one R⁴ group is methyl or ethyl, while the other R⁴ groups are each H; and wherein A-Z is O—Me. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and wherein A-Z is O—Me. In some embodiments, each of the R¹, R², and R⁴ groups are H; and wherein A-Z is O—Me.

In some embodiments, one R¹ group is methyl or ethyl, while the other R¹ groups are each H; R³ is methyl; and wherein A-Z is O—Me. In some embodiments, one R 1 is methyl or ethyl, while the other R¹ groups are each H; one R² group is methyl or ethyl, while the other R² groups are each H; R³ is methyl; and wherein A-Z is O—Me. In some embodiments, one R² is methyl or ethyl, while the other R² groups are each H; one R⁴ group is methyl or ethyl, while the other R⁴ groups are each H; R³ is methyl; and wherein A-Z is O—Me. In some embodiments, one R¹ is methyl or ethyl, while the other R¹ groups are each H; one R⁴ group is methyl or ethyl, while the other R⁴ groups are each H; R³ is methyl; and wherein A-Z is O—Me. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; R³ is methyl; and wherein A-Z is O—Me. In some embodiments, each of the R¹, R², and R⁴ groups are H; R³ is methyl; and wherein A-Z is O—Me.

In some embodiments, one R¹ group is methyl or ethyl, while the other R¹ groups are each H. In some embodiments, one R¹ is methyl or ethyl, while the other R¹ groups are each H; one R² group is methyl or ethyl, while the other R² groups are each H; and where X and Y together form a may form a 5- or 6-membered saturated or unsaturated cyclic or heterocyclic ring optionally substituted with one or more lower alkyl groups. In some embodiments, one R² is methyl or ethyl, while the other R² groups are each H; and one R⁴ group is methyl or ethyl, while the other R⁴ groups are each H; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; and one R⁴ group is methyl or ethyl, while the other R⁴ groups are each H; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and where X and Y together form a may form a 5- or 6-membered saturated or unsaturated cyclic or heterocyclic ring optionally substituted with one or more lower alkyl groups. In some embodiments, each of the R¹, R², and R⁴ groups are H; and where X and Y together form a may form a 5- or 6-membered saturated or unsaturated cyclic or heterocyclic ring optionally substituted with one or more lower alkyl groups.

In some embodiments, one R¹ group is methyl or ethyl, while the other R¹ groups are each H; wherein A is O; and where X and Y together form a may form a 5- or 6-membered saturated or unsaturated cyclic or heterocyclic ring optionally substituted with one or more lower alkyl groups. In some embodiments, one R 1 is methyl or ethyl, while the other R¹ groups are each H; one R² group is methyl or ethyl, while the other R² groups are each H; wherein A is O; and where X and Y together form a may form a 5- or 6-membered saturated or unsaturated cyclic or heterocyclic ring optionally substituted with one or more lower alkyl groups. In some embodiments, one R² is methyl or ethyl, while the other R² groups are each H; one R⁴ group is methyl or ethyl, while the other R⁴ groups are each H; wherein A is O; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R⁴ group is methyl or ethyl, while the other R⁴ groups are each H; wherein A is O; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; wherein A is O; and where X and Y together form a may form a 5- or 6-membered saturated or unsaturated cyclic or heterocyclic ring optionally substituted with one or more lower alkyl groups. In some embodiments, each of the R¹, R², and R⁴ groups are H; A is O; and where X and Y together form a may form a 5- or 6-membered saturated or unsaturated cyclic or heterocyclic ring optionally substituted with one or more lower alkyl groups.

In some embodiments, one R¹ group is methyl or ethyl, while the other R¹ groups are each H; wherein A is ═O; and where X and Y together form a may form a 5- or 6-membered saturated or unsaturated cyclic or heterocyclic ring optionally substituted with one or more lower alkyl groups. In some embodiments, one R 1 is methyl or ethyl, while the other R¹ groups are each H; one R² group is methyl or ethyl, while the other R² groups are each H; wherein A is ═O; and where X and Y together form a may form a 5- or 6-membered saturated or unsaturated cyclic or heterocyclic ring optionally substituted with one or more lower alkyl groups. In some embodiments, one R² is methyl or ethyl, while the other R² groups are each H; one R⁴ group is methyl or ethyl, while the other R⁴ groups are each H; wherein A is ═O; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R⁴ group is methyl or ethyl, while the other R⁴ groups are each H; wherein A is ═O; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; wherein A is ═O; and where X and Y together form a may form a 5- or 6-membered saturated or unsaturated cyclic or heterocyclic ring optionally substituted with one or more lower alkyl groups. In some embodiments, each of the R¹, R², and R⁴ groups are H; A is ═O; and where X and Y together form a may form a 5- or 6-membered saturated or unsaturated cyclic or heterocyclic ring optionally substituted with one or more lower alkyl groups.

In some embodiments, one R¹ group is methyl or ethyl, while the other R¹ groups are each H; R³ is methyl; wherein A is ═O; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R² group is methyl or ethyl, while the other R² groups are each H; R³ is methyl; wherein A is ═O; and where X and Y together form a may form a 5- or 6-membered saturated or unsaturated cyclic or heterocyclic ring optionally substituted with one or more lower alkyl groups. In some embodiments, one R² is methyl or ethyl, while the other R² groups are each H; one R⁴ group is methyl or ethyl, while the other R⁴ groups are each H; R³ is methyl; wherein A is ═O; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R⁴ group is methyl or ethyl, while the other R⁴ groups are each H R³ is methyl; wherein A is ═O; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; R³ is methyl; wherein A is ═O; and where X and Y together form a may form a 5- or 6-membered saturated or unsaturated cyclic or heterocyclic ring optionally substituted with one or more lower alkyl groups. In some embodiments, each of the R¹, R², and R⁴ groups are H; R³ is methyl; A is ═O; and where X and Y together form a may form a 5- or 6-membered saturated or unsaturated cyclic or heterocyclic ring optionally substituted with one or more lower alkyl groups.

In some embodiments, one R¹ group is methyl or ethyl, while the other R¹ groups are each H; wherein A-Z is O—Me; and where X and Y together form a may form a 5- or 6-membered saturated or unsaturated cyclic or heterocyclic ring optionally substituted with one or more lower alkyl groups. In some embodiments, one R 1 is methyl or ethyl, while the other R¹ groups are each H; one R² group is methyl or ethyl, while the other R² groups are each H; wherein A-Z is O—Me; and where X and Y together form a may form a 5- or 6-membered saturated or unsaturated cyclic or heterocyclic ring optionally substituted with one or more lower alkyl groups. In some embodiments, one R² is methyl or ethyl, while the other R² groups are each H; one R⁴ group is methyl or ethyl, while the other R⁴ groups are each H; wherein A-Z is O—Me; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R⁴ group is methyl or ethyl, while the other R⁴ groups are each H; wherein A-Z is O—Me; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; wherein A-Z is O—Me; and where X and Y together form a may form a 5- or 6-membered saturated or unsaturated cyclic or heterocyclic ring optionally substituted with one or more lower alkyl groups. In some embodiments, each of the R¹, R², and R⁴ groups are H; wherein A-Z is O—Me; and where X and Y together form a may form a 5- or 6-membered saturated or unsaturated cyclic or heterocyclic ring optionally substituted with one or more lower alkyl groups.

In some embodiments, one R¹ group is methyl or ethyl, while the other R¹ groups are each H; R³ is methyl; wherein A-Z is O—Me; and where X and Y together form a may form a 5- or 6-membered saturated or unsaturated cyclic or heterocyclic ring optionally substituted with one or more lower alkyl groups. In some embodiments, one R 1 is methyl or ethyl, while the other R¹ groups are each H; one R² group is methyl or ethyl, while the other R² groups are each H; R³ is methyl; wherein A-Z is O—Me; and where X and Y together form a may form a 5- or 6-membered saturated or unsaturated cyclic or heterocyclic ring optionally substituted with one or more lower alkyl groups. In some embodiments, one R² is methyl or ethyl, while the other R² groups are each H; one R⁴ group is methyl or ethyl, while the other R⁴ groups are each H; R³ is methyl; wherein A-Z is O—Me; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R⁴ group is methyl or ethyl, while the other R⁴ groups are each H; R³ is methyl; wherein A-Z is O—Me; and where X and Y together form a may form a 5- or 6-membered saturated or unsaturated cyclic or heterocyclic ring optionally substituted with one or more lower alkyl groups. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; R³ is methyl; wherein A-Z is O—Me; and where X and Y together form a may form a 5- or 6-membered saturated or unsaturated cyclic or heterocyclic ring optionally substituted with one or more lower alkyl groups. In some embodiments, each of the R¹, R², and R⁴ groups are H; R³ is methyl; wherein A-Z is O—Me; and where X and Y together form a may form a 5- or 6-membered saturated or unsaturated cyclic or heterocyclic ring optionally substituted with one or more lower alkyl groups.

In some embodiments, the compounds of the present disclosure have the structure of any one of Formulas (IIA), (IIB), and (IIC):

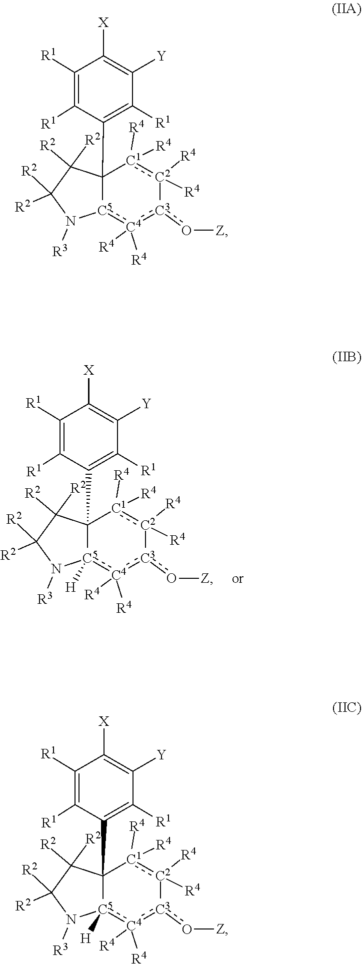

wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
X and Y are each independently —OH, —O-alkyl, —N-alkyl, —S-alkyl, or where X and Y together may form a 5- or 6-membered saturated or unsaturated cyclic or heterocyclic ring optionally substituted with one or more lower alkyl or lower alkoxy groups;
Z is H, a C₁-C₄ alkyl group, or one or more pairs of electrons; and
where the bonds between C¹ and C², between C³ and C⁴, between C⁴ and C⁵, and/or between C³ and —O—Z may be single bonds or double bonds.

In some embodiments, X and Y together form a may form a 5- or 6-membered saturated or unsaturated cyclic or heterocyclic ring optionally substituted with one or more lower alkyl groups. In some embodiments, X and Y together form a may form a 5- or 6-membered saturated or unsaturated cyclic or heterocyclic ring optionally substituted with one or more lower alkyl groups; and where Z is H or methyl. In some embodiments, X and Y together form a may form a 5- or 6-membered saturated or unsaturated cyclic or heterocyclic ring optionally substituted with one or more lower alkyl groups. In some embodiments, X and Y together form a may form a 5- or 6-membered saturated or unsaturated cyclic or heterocyclic ring optionally substituted with one or more lower alkyl groups; where Z is H or methyl; and where R³ is methyl.

In some embodiments, one R¹ group is methyl or ethyl, while the other R¹ groups are each H. In some embodiments, one R 1 is methyl or ethyl, while the other R¹ groups are each H; and one R² group is methyl or ethyl, while the other R² groups are each H. In some embodiments, one R² is methyl or ethyl, while the other R² groups are each H; and one R⁴ group is methyl or ethyl, while the other R⁴ groups are each H. In some embodiments, one R¹ is methyl or ethyl, while the other R¹ groups are each H; and one R⁴ group is methyl or ethyl, while the other R⁴ groups are each H. In some embodiments, one R′ is methyl or ethyl, while the other R¹ groups are each H; one R² group is methyl or ethyl, while the other R² groups are each H; one R⁴ group is methyl or ethyl, while the other R⁴ groups are each H. In some embodiments, each of the R¹, R², and R⁴ groups are H.

In some embodiments, one R¹ group is methyl or ethyl, while the other R¹ groups are each H; and Z is methyl. In some embodiments, one R 1 is methyl or ethyl, while the other R¹ groups are each H; one R² group is methyl or ethyl, while the other R² groups are each H; and Z is methyl. In some embodiments, one R² is methyl or ethyl, while the other R² groups are each H; one R⁴ group is methyl or ethyl, while the other R⁴ groups are each H; and Z is methyl. In some embodiments, one R¹ is methyl or ethyl, while the other R¹ groups are each H; one R⁴ group is methyl or ethyl, while the other R⁴ groups are each H; and Z is methyl. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and Z is methyl.

In some embodiments, Z is H or methyl; and R³ is methyl. In some embodiments, Z is methyl and R³ is methyl.

In some embodiments, the compounds of the present disclosure have the structure of any one of Formulas (IIIA), (IIIB), or (IIIC):

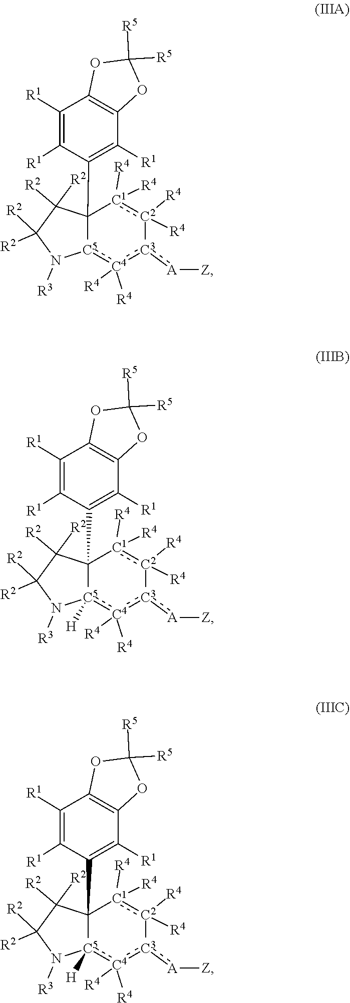

wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;

R³ is —CH₃ or —CH₂—CH₃;

each R⁴ is independently a substituted or unsubstituted C₁-C₄ alkyl group;

A is O or S;

Z is H, a C₁-C₄ alkyl group, or one or more pairs of electrons; and

each R⁵ is independently H or a C₁-C₄ alkyl group; and

where the bonds between C¹ and C², between C³ and C⁴, between C⁴ and C⁵, and/or between C³ and A may be single bonds or double bonds.

In some embodiments, A is O. In some embodiments, A is O and Z is H. In some embodiments, Z is methyl or ethyl. In some embodiments, Z is methyl. In some embodiments, A is O and Z is methyl or ethyl. In some embodiments, A is O and Z is methyl. In some embodiments, A is O, Z is methyl, and R³ is methyl.

In some embodiments, at least one R⁵ group is selected from H, methyl, or ethyl. In some embodiments, one R⁵ group is H; and the other R⁵ group is methyl or ethyl. In some embodiments, both R⁵ groups are selected from methyl or ethyl. In some embodiments, both R⁵ groups are H.

In some embodiments, at least one R⁵ group is H, A is O, and Z is methyl or ethyl. In some embodiments, at least one R⁵ group is H, A is O, and Z is H or methyl. In some embodiments, at least one R⁵ group is H, A is O, R³ is methyl, and Z is methyl or ethyl. In some embodiments, at least one R⁵ group is H, A is O, R³ is methyl, and Z is H or methyl.

In some embodiments, one R¹ group is methyl or ethyl, while the other R¹ groups are each H. In some embodiments, one R 1 is methyl or ethyl, while the other R¹ groups are each H; and one R² group is methyl or ethyl, while the other R² groups are each H. In some embodiments, one R² is methyl or ethyl, while the other R² groups are each H; and one R⁴ group is methyl or ethyl, while the other R⁴ groups are each H. In some embodiments, one R¹ is methyl or ethyl, while the other R¹ groups are each H; and one R⁴ group is methyl or ethyl, while the other R⁴ groups are each H. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H. In some embodiments, each of the R¹, R², and R⁴ groups are H. In some embodiments, each of the R¹, R², and R⁴ groups are H; and wherein at least one R⁵ group is methyl. In some embodiments, each of the R¹, R², and R⁴ groups are H; and wherein at least one R⁵ group is H. In some embodiments, each of the R¹, R², and R⁴ groups are H; and wherein both R⁵ groups are methyl. In some embodiments, each of the R¹, R², and R⁴ groups are H; and wherein both R⁵ groups are H.

In some embodiments, one R¹ group is methyl or ethyl, while the other R¹ groups are each H; and wherein A is O. In some embodiments, one R 1 is methyl or ethyl, while the other R¹ groups are each H; one R² group is methyl or ethyl, while the other R² groups are each H; and wherein A is O. In some embodiments, one R² is methyl or ethyl, while the other R² groups are each H; one R⁴ group is methyl or ethyl, while the other R⁴ groups are each H; and wherein A is O. In some embodiments, one R¹ is methyl or ethyl, while the other R¹ groups are each H; one R⁴ group is methyl or ethyl, while the other R⁴ groups are each H; and wherein A is O. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and wherein A is O. In some embodiments, each of the R¹, R², and R⁴ groups are H; and A is O. In some embodiments, each of the R¹, R², and R⁴ groups are H; wherein at least one R⁵ group is methyl; and wherein A is O. In some embodiments, each of the R¹, R², and R⁴ groups are H; wherein at least one R⁵ group is H; and wherein A is O. In some embodiments, each of the R¹, R², and R⁴ groups are H; wherein both R⁵ groups are methyl; and wherein A is O. In some embodiments, each of the R¹, R², and R⁴ groups are H; wherein both R⁵ groups are H; and wherein A is O.

In some embodiments, one R¹ group is methyl or ethyl, while the other R¹ groups are each H; and wherein A is ═O. In some embodiments, one R 1 is methyl or ethyl, while the other R¹ groups are each H; one R² group is methyl or ethyl, while the other R² groups are each H; and wherein A is ═O. In some embodiments, one R² is methyl or ethyl, while the other R² groups are each H; one R⁴ group is methyl or ethyl, while the other R⁴ groups are each H; and wherein A is ═O. In some embodiments, one R¹ is methyl or ethyl, while the other R¹ groups are each H; one R⁴ group is methyl or ethyl, while the other R⁴ groups are each H; and wherein A is ═O. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and wherein A is ═O. In some embodiments, each of the R¹, R², and R⁴ groups are H; and A is ═O. In some embodiments, each of the R¹, R², and R⁴ groups are H; wherein at least one R⁵ group is methyl; and wherein A is ═O. In some embodiments, each of the R¹, R², and R⁴ groups are H; wherein at least one R⁵ group is H; and wherein A is ═O. In some embodiments, each of the R¹, R², and R⁴ groups are H; wherein both R⁵ groups are methyl; and wherein A is ═O. In some embodiments, each of the R¹, R², and R⁴ groups are H; wherein both R⁵ groups are H; and wherein A is ═O.

In some embodiments, one R¹ group is methyl or ethyl, while the other R¹ groups are each H; R³ is methyl; and wherein A is ═O. In some embodiments, one R 1 is methyl or ethyl, while the other R¹ groups are each H; one R² group is methyl or ethyl, while the other R² groups are each H; R³ is methyl; and wherein A is ═O. In some embodiments, one R² is methyl or ethyl, while the other R² groups are each H; one R⁴ group is methyl or ethyl, while the other R⁴ groups are each H; R³ is methyl; and wherein A is ═O. In some embodiments, one R¹ is methyl or ethyl, while the other R¹ groups are each H; one R⁴ group is methyl or ethyl, while the other R⁴ groups are each H R³ is methyl; and wherein A is ═O. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; R³ is methyl; and wherein A is =O. In some embodiments, each of the R¹, R², and R⁴ groups are H; R³ is methyl; and A is ═O. In some embodiments, each of the R¹, R², and R⁴ groups are H; wherein at least one R⁵ group is methyl; R³ is methyl; and wherein A is ═O. In some embodiments, each of the R¹, R², and R⁴ groups are H; wherein at least one R⁵ group is H; R³ is methyl; and wherein A is ═O. In some embodiments, each of the R¹, R², and R⁴ groups are H; wherein both R⁵ groups are methyl; R³ is methyl; and wherein A is ═O. In some embodiments, each of the R¹, R², and R⁴ groups are H; wherein both R⁵ groups are H; R³ is methyl; and wherein A is ═O.

In some embodiments, one R¹ group is methyl or ethyl, while the other R¹ groups are each H; and wherein A-Z is O—Me. In some embodiments, one R 1 is methyl or ethyl, while the other R¹ groups are each H; one R² group is methyl or ethyl, while the other R² groups are each H; and wherein A-Z is O—Me. In some embodiments, one R² is methyl or ethyl, while the other R² groups are each H; one R⁴ group is methyl or ethyl, while the other R⁴ groups are each H; and wherein A-Z is O—Me. In some embodiments, one R¹ is methyl or ethyl, while the other R¹ groups are each H; one R⁴ group is methyl or ethyl, while the other R⁴ groups are each H; and wherein A-Z is O—Me. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; and wherein A-Z is O—Me. In some embodiments, each of the R¹, R², and R⁴ groups are H; and wherein A-Z is O—Me. In some embodiments, each of the R¹, R², and R⁴ groups are H; wherein at least one R⁵ group is methyl; and wherein A-Z is O—Me. In some embodiments, each of the R¹, R², and R⁴ groups are H; wherein at least one R⁵ group is H; and wherein A-Z is O—Me. In some embodiments, each of the R¹, R², and R⁴ groups are H; wherein both R⁵ groups are methyl; and wherein A-Z is O—Me. In some embodiments, each of the R¹, R², and R⁴ groups are H; wherein both R⁵ groups are H; and wherein A-Z is O—Me.

In some embodiments, one R¹ group is methyl or ethyl, while the other R¹ groups are each H; R³ is methyl; and wherein A-Z is O—Me. In some embodiments, one R 1 is methyl or ethyl, while the other R¹ groups are each H; one R² group is methyl or ethyl, while the other R² groups are each H; R³ is methyl; and wherein A-Z is O—Me. In some embodiments, one R² is methyl or ethyl, while the other R² groups are each H; one R⁴ group is methyl or ethyl, while the other R⁴ groups are each H; R³ is methyl; and wherein A-Z is O—Me. In some embodiments, one R¹ is methyl or ethyl, while the other R¹ groups are each H; one R⁴ group is methyl or ethyl, while the other R⁴ groups are each H; R³ is methyl; and wherein A-Z is O—Me. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H; R³ is methyl; and wherein A-Z is O—Me. In some embodiments, each of the R¹, R², and R⁴ groups are H; R³ is methyl; and wherein A-Z is O—Me. In some embodiments, each of the R¹, R², and R⁴ groups are H; wherein at least one R⁵ group is methyl; R³ is methyl; and wherein A-Z is O—Me. In some embodiments, each of the R¹, R², and R⁴ groups are H; wherein at least one R⁵ group is H; R³ is methyl; and wherein A-Z is O—Me. In some embodiments, each of the R¹, R², and R⁴ groups are H; wherein both R⁵ groups are methyl; R³ is methyl; and wherein A-Z is O—Me. In some embodiments, each of the R¹, R², and R⁴ groups are H; wherein both R⁵ groups are H; R³ is methyl; and wherein A-Z is O—Me.

In some embodiments, the compounds of the present disclosure have the structure of any one of Formulas (IVA), (IVB), or (IVC):

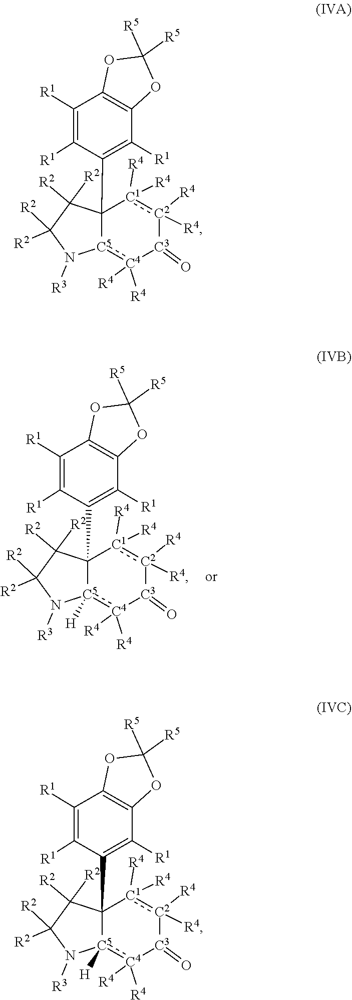

wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
A is O or S;
R⁵ is a C₁-C₄ alkyl; and
where the bonds between C¹ and C² and/or between C⁴ and C⁵ may be single bonds or double bonds.

In some embodiments, at least one R⁵ group is selected from H, methyl, or ethyl. In some embodiments, one R⁵ group is H; and the other R⁵ group is methyl or ethyl. In some embodiments, both R⁵ groups are selected from methyl or ethyl. In some embodiments, both R⁵ groups are H.

In some embodiments, A is O. In some embodiments, A is O and Z is H. In some embodiments, A is O and Z is methyl or ethyl. In some embodiments, A is O and Z is methyl. In some embodiments, at least one R⁵ group is H, A is O, and Z is methyl or ethyl. In some embodiments, at least one R⁵ group is H, A is O, and Z is H or methyl. In some embodiments, at least one R⁵ group is H, A is O, R³ is methyl, and Z is methyl or ethyl. In some embodiments, at least one R⁵ group is H, A is O, R³ is methyl, and Z is H or methyl.

In some embodiments, one R¹ group is methyl or ethyl, while the other R¹ groups are each H. In some embodiments, one R 1 is methyl or ethyl, while the other R¹ groups are each H; and one R² group is methyl or ethyl, while the other R² groups are each H. In some embodiments, one R² is methyl or ethyl, while the other R² groups are each H; and one R⁴ group is methyl or ethyl, while the other R⁴ groups are each H. In some embodiments, one R¹ is methyl or ethyl, while the other R¹ groups are each H; and one R⁴ group is methyl or ethyl, while the other R⁴ groups are each H. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H. In some embodiments, each of the R′, R², and R⁴ groups are H. In some embodiments, each of the R¹, R², and R⁴ groups are H; and wherein at least one R⁵ group is methyl. In some embodiments, each of the R¹, R², and R⁴ groups are H; and wherein at least one R⁵ group is H. In some embodiments, each of the R¹, R², and R⁴ groups are H; and wherein both R⁵ groups are methyl. In some embodiments, each of the R¹, R², and R⁴ groups are H; and wherein both R⁵ groups are H.

In some embodiments, the compounds of the present disclosure have the structure of any one of Formulas (VA), (VB), (VC), (VD), (VE), or (VF):

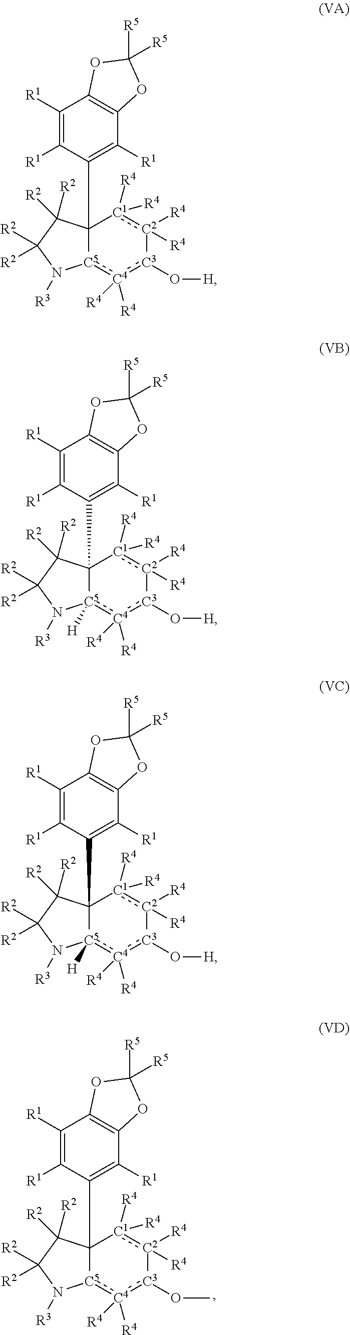

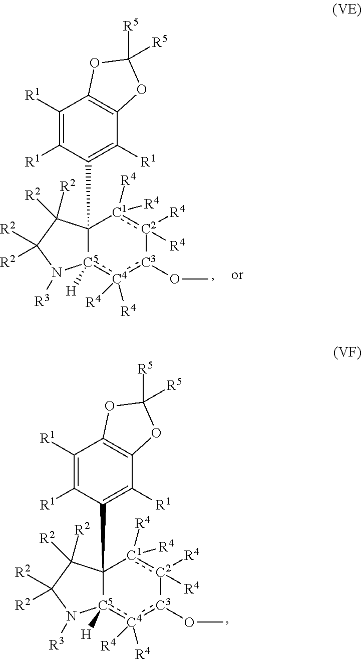

wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
A is O or S;
R⁵ is a C₁-C₄ alkyl; and
where any one of the bonds between C¹ and C², C³ and C⁴, and/or C⁴ and C⁵ may be single bonds or double bonds.

In some embodiments, one R¹ group is methyl or ethyl, while the other R¹ groups are each H. In some embodiments, one R¹ is methyl or ethyl, while the other R¹ groups are each H; and one R² group is methyl or ethyl, while the other R² groups are each H. In some embodiments, one R² is methyl or ethyl, while the other R² groups are each H; and one R⁴ group is methyl or ethyl, while the other R⁴ groups are each H. In some embodiments, one R¹ is methyl or ethyl, while the other R¹ groups are each H; and one R⁴ group is methyl or ethyl, while the other R⁴ groups are each H. In some embodiments, one R¹ is methyl or ethyl, while the other R¹ groups are each H; one R² group is methyl or ethyl, while the other R² groups are each H; one R⁴ group is methyl or ethyl, while the other R⁴ groups are each H. In some embodiments, each of the R¹, R², and R⁴ groups are H. In some embodiments, each of the R¹, R², and R⁴ groups are H; and wherein at least one R⁵ group is methyl. In some embodiments, each of the R¹, R², and R⁴ groups are H; and wherein at least one R⁵ group is H. In some embodiments, each of the R¹, R², and R⁴ groups are H; and wherein both R⁵ groups are methyl. In some embodiments, each of the R¹, R², and R⁴ groups are H; and wherein both R⁵ groups are H.

In some embodiments, the compounds of the present disclosure have the structure of any one of Formulas (VIA), (VIB), or (VIC):

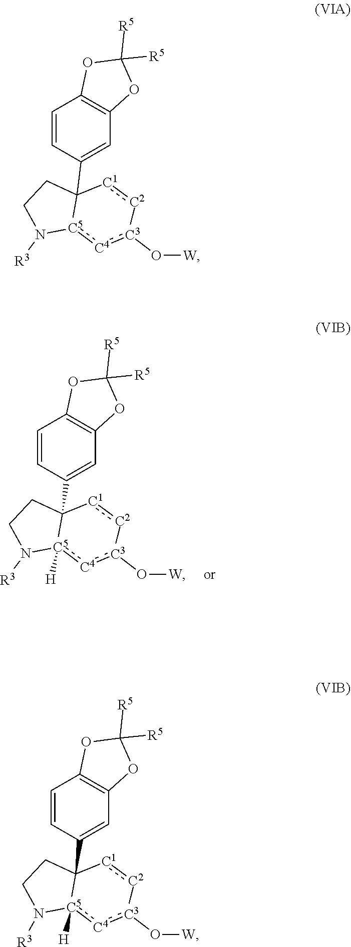

wherein
R³ is —CH₃ or —CH₂—CH₃;
each R⁵ is independently H or a C₁ to C₄ alkyl group; and
where the bonds between C¹ and C² and/or between C⁴ and C⁵ may be single bonds or double bonds.

In some embodiments, R³ is methyl. In some embodiments, R³ is ethyl.

In some embodiments, the compounds of the present disclosure have the structure of Formula (VII):

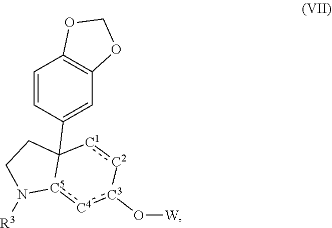

wherein
R³ is —CH₃ or —CH₂—CH₃;
W is H or C₁-C₄ alkyl group; and
where any of the bonds between C¹ and C², C³ and C⁴, or C⁴ and C⁵ may be a single bond or a double bond.

In some embodiments, R³ is methyl. In some embodiments, R³ is ethyl.

In some embodiments, W is methyl and R³ is methyl. In some embodiments, W is H and R³ is methyl.

In some embodiments, the compounds of the present disclosure have the structure of any one of the Formulas (VIIIA), (VIIIB), or (VIIIC):

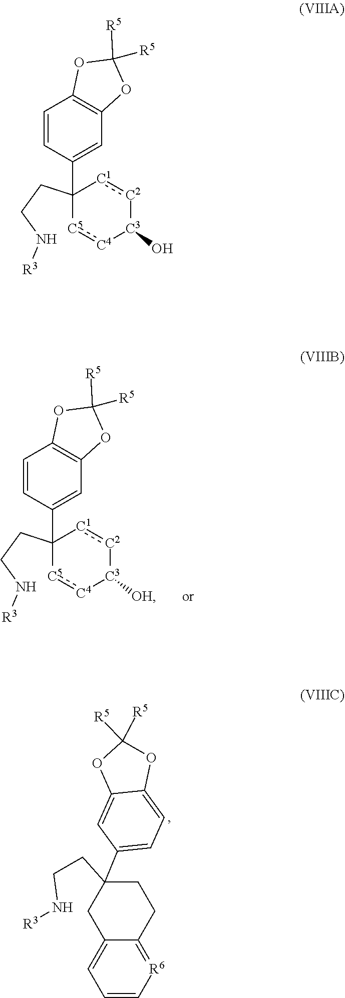

wherein
R³ is methyl or ethyl;
each R⁵ is independently H or a C₁-C₄ alkyl group;
R⁶ is O, NH, NCH₃, or S; and
wherein the bonds between C¹ and C² and/or between C⁴ and C⁵ may be single bonds or double bonds.

In some embodiments, the compounds of the present disclosure are selected from:

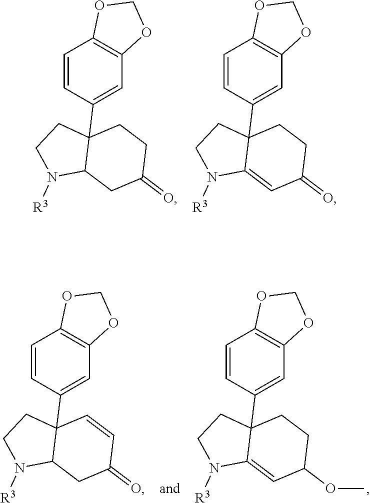

where R³ is methyl or ethyl. In some embodiments. R³ is methyl.

In some embodiments, the compounds of the present disclosure are selected from:

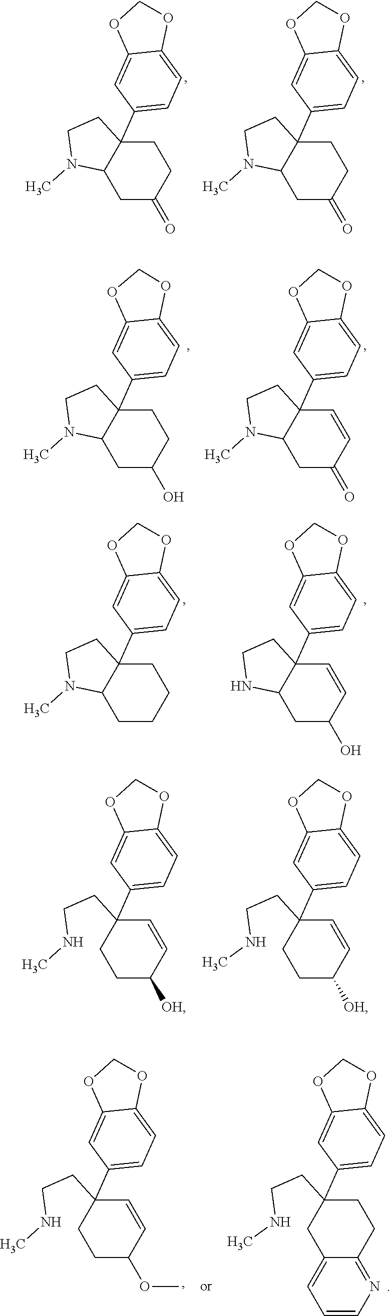

### Synthesis

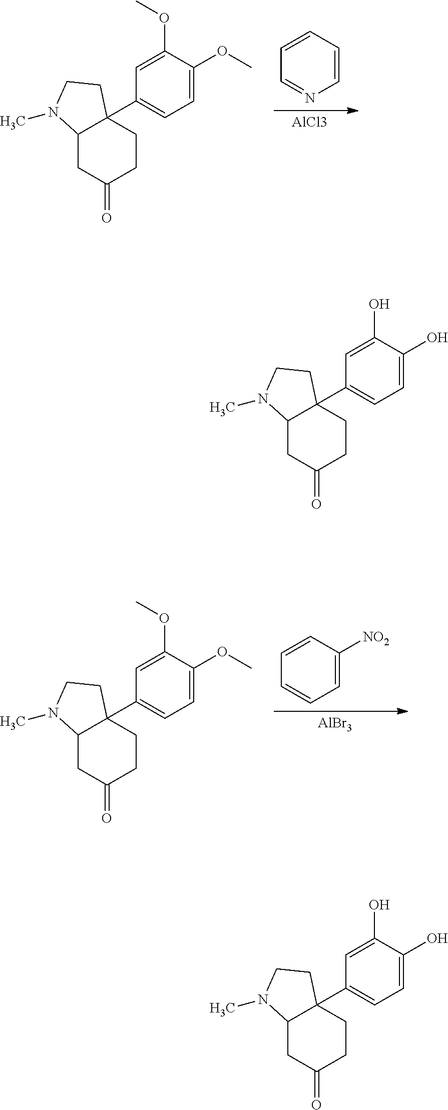

Mesembrine Demethylation with Pyridine and AlCl₃

Anhydrous AlCl₃ was suspended in a solution of mesembrine in methylene chloride in an apparatus protected from atmospheric moisture. While stirring briskly and cooling to maintain a cool temperature while pyridine was added slowly. The reaction was heated to reflux and maintained at that temperature with stirring for 24 hours. The solution was cooled, and the product was hydrolyzed while stirring by the addition of dilute HCl until the mixture was acidic. The methylene chloride layer contained most of the small amount of mesembrine, and the product was in the aqueous phase. Extraction of the aqueous phase with ether, followed by evaporation of the ether, left the desired product.

Mesembrine Demethylation with Nitrobenzene and AlBr₃₂

A solution of mesembrine in itrobenzene was treated with a solution anhydrous aluminium bromide in nitrobenzene. The solution was heated and then let cool to room temperature and allowed to stand at room temperature for 30 minutes. The mixture was cooled and added to water containing a little HCl. The mixture is extracted with ether, and the ether is then extracted with 5% NaOH. The hydroxide solution is washed with ether and then acidified with dilute sulfuric acid. The acidic solution is extracted with ether, dried, and then distilled off to leave the desired product

Mesembrine Demethylation with Lithium Chloride in DMF

A solution of mesembrine and lithium chloride in N,N-dimethylformamide was brought to reflux. Further lithium chloride was added. After cooling, toluene was added, and the resultant precipitate was filtered off with suction and extracted with toluene. The organic extracts were combined and concentrated on a rotary evaporator. After flash chromatography on silica gel the product was obtained.

### Methyleneation Reactions

Demthylated mesembrine and derivatives react with a dihalomethane to form a methylenedioxybenzene (1,3-benzodioxole) derivative under the influence of a base as a catalyst.

The dihalomethane used in the reaction can either be methylene chloride, methylene bromide or methylene iodide. Mixed halomethanes can also be used, such as bromochloromethane. The reactivity is the highest with the iodo derivatives and lowest with the chlorides. The base, most commonly sodium hydroxide, is used to deprotonate the phenols to diphenoxide dianions, making them much more reactive toward dihalomethanes.

### Formulations

The present disclosure is also directed to a composition comprising one or more of the compounds of the present disclosure described herein and one or more pharmaceutically acceptable carriers or excipients. In some embodiments, the present disclosure is directed to formulations suitable for administration to a mammal. In some embodiments, the formulations are provided for administration to a human subject.

Non-limiting examples of pharmaceutically acceptable carriers or excipients include penetration or permeation enhancers, plasticizers, gelling agents, fluid carriers, oils, tackifiers, cohesion-promoting additives, stabilizers, fillers, colorants, dyes, UV-absorbing compounds, buffers, preservatives, emulsifiers, antimicrobial and/or antifungal agents, wetting agents, antioxidants and similar additives. A person skilled in the art will be able to select the suitable carriers excipients or mixtures of carriers or excipients for the desired formulations. In general, the amount of any pharmaceutically acceptable excipient, carrier, and/or additive included within any formulations may vary depending on the desired effect, route of administration, form of the final composition. In general, however, a total amount of pharmaceutically acceptable excipients, carriers, and/or additives formulated with the formulations of the present disclosure may range from about 1% to about 99% by total weight of the formulations. In some embodiments, a total amount of pharmaceutically acceptable excipients, carriers, and/or additives formulated with the formulations of the present disclosure may range from about 1% to about 98% by total weight of the formulations. In some embodiments, a total amount of pharmaceutically acceptable excipients, carriers, and/or additives formulated with the formulations of the present disclosure may range from about 1% to about 97% by total weight of the formulations. In some embodiments, a total amount of pharmaceutically acceptable excipients, carriers, and/or additives formulated with the formulations of the present disclosure may range from about 1% to about 96% by total weight of the formulations. In some embodiments, a total amount of pharmaceutically acceptable excipients, carriers, and/or additives formulated with the formulations of the present disclosure may range from about 1% to about 95% by total weight of the formulations. In some embodiments, a total amount of pharmaceutically acceptable excipients, carriers, and/or additives formulated with the formulations of the present disclosure may range from about 1% to about 94% by total weight of the formulations. In some embodiments, a total amount of pharmaceutically acceptable excipients, carriers, and/or additives formulated with the formulations of the present disclosure may range from about 1% to about 93% by total weight of the formulations. In some embodiments, a total amount of pharmaceutically acceptable excipients, carriers, and/or additives formulated with the formulations of the present disclosure may range from about 1% to about 92% by total weight of the formulations. In some embodiments, a total amount of pharmaceutically acceptable excipients, carriers, and/or additives formulated with the formulations of the present disclosure may range from about 1% to about 91% by total weight of the formulations. \

In other embodiments, the total amount of pharmaceutically acceptable excipients, carriers, and/or additives formulated with the formulations of the present disclosure may range from about 1% to about 90% by total weight of the formulation. In some embodiments, a total amount of pharmaceutically acceptable excipients, carriers, and/or additives formulated with the formulations of the present disclosure may range from about 1% to about 88% by total weight of the formulations. In some embodiments, a total amount of pharmaceutically acceptable excipients, carriers, and/or additives formulated with the formulations of the present disclosure may range from about 1% to about 86% by total weight of the formulations. In some embodiments, a total amount of pharmaceutically acceptable excipients, carriers, and/or additives formulated with the formulations of the present disclosure may range from about 1% to about 84% by total weight of the formulations. In some embodiments, a total amount of pharmaceutically acceptable excipients, carriers, and/or additives formulated with the formulations of the present disclosure may range from about 1% to about 82% by total weight of the formulations. In other embodiments, the total amount of pharmaceutically acceptable excipients, carriers, and/or additives formulated with the formulations of the present disclosure may range from about 1% to about 80% by total weight of the formulation. In some embodiments, a total amount of pharmaceutically acceptable excipients, carriers, and/or additives formulated with the formulations of the present disclosure may range from about 1% to about 78% by total weight of the formulations. In some embodiments, a total amount of pharmaceutically acceptable excipients, carriers, and/or additives formulated with the formulations of the present disclosure may range from about 1% to about 76% by total weight of the formulations. In some embodiments, a total amount of pharmaceutically acceptable excipients, carriers, and/or additives formulated with the formulations of the present disclosure may range from about 1% to about 74% by total weight of the formulations. In some embodiments, a total amount of pharmaceutically acceptable excipients, carriers, and/or additives formulated with the formulations of the present disclosure may range from about 1% to about 72% by total weight of the formulations. In other embodiments, the total amount of pharmaceutically acceptable excipients, carriers, and/or additives formulated with the formulations of the present disclosure may range from about 1% to about 70% by total weight of the formulation.

Examples of pharmaceutically acceptable binders include, but are not limited to, starches, celluloses, and derivatives thereof. Examples of celluloses and their derivatives include hydroxypropyl methylcellulose, carboxymethylcellulose, and salts thereof (especially sodium and calcium salts), methylcellulose, hydroxyethyl cellulose, ethyl hydroxyethyl cellulose, hydroxypropyl cellulose (high or low substituted), hydroxylethyl cellulose, microcrystalline cellulose, sucrose, dextrose, corn syrup, polysaccharides, gelatin, and mixtures thereof.

Other examples of binders that may be included in the compositions of the present disclosure include conventional hydrogels formed using water-soluble or water-insoluble gums or resins, with or without known cross-linking agents. The gums or resins include agarose, alginates, alkyl and hydroxyalkyl celluloses, such as hydroxyethyl cellulose and hydroxypropyl cellulose, amylopectin, arabinogalactin, carboxymethyl cellulose, carrageenan, eucheuma, ucoidan, furcellaran, gelatin, guar gum, gum agar, gum arabic, gum ghatti, gum karaya, gum tragacanth, pypenia, keratin laminaran, locust bean gum, pectin, polyacrylamide, poly(acrylic)acid and homologs, polyethylene glycol, poly(ethylene oxide), poly(hydroxyalkyl) methacrylate, polyvinyl alcohol, polyvinylpyrrolidone, propylene glycol alginate, starch and modified analogs, tamarind gum, N-vinyl lactam polysaccharides and xanthan gum. In addition, such hydrogels can be formed by the copolymerization and cross-linking of both hydrophilic and hydrophobic monomers, such as hydroxy-alkyl esters of acrylic acid and methacrylamide, n-vinyl-1-pyrrolidone, alkyl acrylates and methacrylates, vinyl acetate, acrylonitrile and styrene. Other binders suitable for use with the present disclosure include vecgum, higher molecular weight polyglycols, and the like. In particularly useful embodiments, low substituted hydroxypropyl cellulose is used.

Within the context of the present invention, the binder may be present in the final dosage form at a concentration from about 1% to 10% w/w, which includes 1-9%, 1-8%, 1-7%, 1-6%, 1-5%, 1-4%, 1-3%, 1-2%, 2-10%, 2-9%, 2-8%, 2-7%, 2-6%, 2-5%, 2-4%, 2-3%, 3-10%, 3-9%, 3-8%, 3-7%, 3-6%, 3-5%, 3-4%, 4-10%, 4-9%, 4-8%, 4-7%, 4-6%, 4-5%, 5-10%, 5-9%, 5-8%, 5-7%, 5-6%, 6-10%, 6-9%, 6-8%, 6-7%, 7-10%, 7-9%, 7-8%, 8-10%, 8-9%, and 9-10% w/w. In particularly useful embodiments, the binder is present at a concentration of about 3% to 5% w/w.

Examples of pharmaceutically acceptable disintegrants include, but are not limited to, starches clays, celluloses, alginates, gums (e.g., guar), cross-linked polymers (e.g., crospovidone such as Polyplasdone XL and PolyplasdoneXL-10), croscarmellose sodium, (e.g., AC-DI-SOL), croscarmellose calcium, soy polysaccharides, and mixtures thereof. In particularly useful embodiments, crospovidone is used as a disintegrant. Within the context of the present invention, the disintegrant may be present in the final dosage form at a concentration from about 1% to 10% w/w which includes 1-9%, 1-8%, 1-7%, 1-6%, 1-5%, 1-4%, 1-3%, 1-2%, 2-10%, 2-9%, 2-8%, 2-7%, 2-6%, 2-5%, 2-4%, 2-3%, 3-10%, 3-9%, 3-8%, 3-7%, 3-6%, 3-5%, 3-4%, 4-10%, 4-9%, 4-8%, 4-7%, 4-6%, 4-5%, 5-10%, 5-9%, 5-8%, 5-7%, 5-6%, 6-10%, 6-9%, 6-8%, 6-7%, 7-10%, 7-9%, 7-8%, 8-10%, 8-9%, and 9-10% w/w. In particularly useful embodiments, the disintegrant is present at a concentration of about 3% to 5% w/w. Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose, glyceryl behenate, stearic acid, hydrogenated castor oil, glyceryl monostearate, sodium stearyl fumarate, and mixtures thereof. In particularly useful embodiments, magnesium stearate is used as a lubricant and colloidal silica is used as a glidant.

Within the context of the present invention, the lubricant may be present in the final dosage form at a concentration from about 0.01% to 5% w/w, which also includes 0.05-5%, 0.5-4%, 0.5-3%, 0.5-2%, 0.5-1%, 1-5%, 1-4%, 1-3%, 1-2%, 2-5%, 2-4%, 2-4%, 3-5%, 3-4%, and 4-5%. In some embodiments, the lubricant is present at a concentration of about 0.5% to 1% w/w. In some embodiments, the glidant may be present in the final dosage form at a concentration from about O. 01% to 5% w/w, which also includes 0.05-5%, 0.5-4%, 0.5-3%, 0.5-2%, 0.5-1%, 1-5%, 1-4%, 1-3%, 1-2%, 2-5%, 2-4%, 2-4%, 3-5%, 3-4%, and 4-5%. In particularly useful embodiments, the glidant is present at a concentration of about 0.5% to 1% w/w.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, and mixtures thereof. In particularly useful embodiments, microcrystalline cellulose, powdered cellulose, sorbitol, sucrose, or mixtures thereof are used. In other particularly useful embodiments, microcrystalline cellulose is used. In some embodiments, the filler/diluent may be present in the final dosage form at a concentration from about 1% to 10% which includes 1-9%, 1-8%, 1-7%, 1-6%, 1-5%, 1-4%, 1-3%, 1-2%, 2-10%, 2-9%, 2-8%, 2-7%, 2-6%, 2-5%, 2-4%, 2-3%, 3-10%, 3-9%, 3-8%, 3-7%, 3-6%, 3-5%, 3-4%, 4-10%, 4-9%, 4-8%, 4-7%, 4-6%, 4-5%, 5-10%, 5-9%, 5-8%, 5-7%, 5-6%, 6-10%, 6-9%, 6-8%, 6-7%, 7-10%, 7-9%, 7-8%, 8-10%, 8-9%, and 9-10% w/w. In particularly useful embodiments, the filler/diluent is present at a concentration of about 5% to 7% w/w.

Solid oral dosage forms, for example, tablets or capsules, may be further coated with suitable coating agents by methods well-known to a person skilled in the art. Examples of suitable coating agents include, but are not limited to, Opadry AMB-II, Opadry white, Opadry yellow, Opadry red, Opadry black, and Aquarius.

The term “antioxidant” is used herein includes any compound or combination of compounds that prevent, or slow down oxidation of components caused by the damaging reactive oxygen species (ROS). Any of the known antioxidants may be used, including but not limited to tocopherols, phospholipids (PL), phytosterols, phycocyanin, vitamins E, A and C, betacarotene, coenzyme Q10, fatty acids omega-3, omega-6 and w-9, phytoantioxidants such as polyphenols, terpenes as butylated hydroxyanisole (BHA), butylated hydroxytoluene (BHT), propyl gallate, lecithin, sesamin, sesamol, sesamolin, α-tocopherol, γ-tocopherol, salicylic acid, ascorbic acid, ascorbyl palmitate, fumaric acid, malic acid, sodium ascorbate and sodium meta-bisulphite, as well as chelating agents such as disodium EDTA. Pharmaceutically acceptable nutraceutical dietary supplements may also be employed as antioxidants including plants, alga, and lichen and may include one or more extracts of honeybee propolis, red clover, soybean, caper, almond, milk thistle, green tea, pomegranate, orange red, grape seed, bilberry, fo-ti root, ginseng, English ivy, red algae, brown algae, green algae and lichens. Suitable preservatives in the composition of the present invention may include, but not limited to, phenoxyethanol, alkyl para-hydroxybenzoates, wherein the alkyl radical has from 1, 2, 3, 4, 5 or 6 carbon atoms and preferably from 1 to 4 carbon atoms e.g., methyl para-hydroxybenzoate(methylparaben), ethyl para-hydroxybenzoate(ethylparaben), propyl para-hydroxybenzoate(propylparaben), butyl para-hydroxybenzoate(butylparaben), isobutyl para-hydroxybenzoate(isobutylparaben), and their mixtures.

In some embodiments, an amount of a compound of the present disclosure within any formulation ranges from between about 0.5% to about 30% by total weight of the composition. In other embodiments, an amount of a compound of the present disclosure within any composition ranges from between about 1% to about 30% by total weight of the composition. In yet other embodiments, an amount of a compound of the present disclosure within any composition ranges from between about 1% to about 25% by total weight of the composition. In further embodiments, an amount of a compound of the present disclosure within any composition ranges from between about 1% to about 20% by total weight of the composition. In even further embodiments, an amount of a compound of the present disclosure within any composition ranges from between about 1% to about 15% by total weight of the composition. In yet further embodiments, an amount of a compound of the present disclosure within any composition ranges from between about 1% to about 10% by total weight of the composition. In yet even further embodiments, an amount of a compound of the present disclosure within any composition ranges from between about 1% to about 5% by total weight of the composition.

In some embodiments, an amount of a compound of the present disclosure within any formulation ranges from between about 0.000001% to about 5% by total weight of the composition. In other embodiments, an amount of a compound of the present disclosure within any composition ranges from between about 0.000001% to about 3% by total weight of the composition. In yet other embodiments, an amount of a compound of the present disclosure within any composition ranges from between about 0.000001% to about 2.5% by total weight of the composition. In further embodiments, an amount of a compound of the present disclosure within any composition ranges from between about 0.00001% to about 2.5% by total weight of the composition. In even further embodiments, an amount of a compound of the present disclosure within any composition ranges from between about 0.0001% to about 2.5% by total weight of the composition. In yet further embodiments, an amount of a compound of the present disclosure within any composition ranges from between about 0.001% to about 2.5% by total weight of the composition. In yet even further embodiments, an amount of a compound of the present disclosure within any composition ranges from between about 0.01% to about 2.5% by total weight of the composition. In yet even further embodiments, an amount of a compound of the present disclosure within any composition ranges from between about 0.01% to about 2% by total weight of the composition. In yet even further embodiments, an amount of a compound of the present disclosure within any composition ranges from between about 0.01% to about 1.5% by total weight of the composition. In yet even further embodiments, an amount of a compound of the present disclosure within any composition ranges from between about 0.1% to about 1.5% by total weight of the composition. In yet even further embodiments, an amount of a compound of the present disclosure within any composition ranges from between about 0.1% to about 1% by total weight of the composition. In yet even further embodiments, an amount of a compound of the present disclosure within any composition ranges from between about 0.1% to about 0.5% by total weight of the composition.

The formulations of the present disclosure may be provided, in general, in the form of discrete units such as hard or soft capsules, tablets, troches or lozenges, each containing a predetermined amount of one or more of the compounds described herein; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

### Methods of Treatment

The present disclosure is also directed to administering therapeutically effective amounts of a pharmaceutical composition comprising one or more of the compounds described herein. In some embodiments, the pharmaceutical compositions administered to a subject to treat depressive states, psychological or psychiatric disorders with an anxiety component, alcohol and drug dependence, bulimia nervosa, PTSD, and/or obsessive-compulsive disorders.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A compound having any one of any one of Formulas (VA), (VB), (VC), (VD), (VE), or (VF): 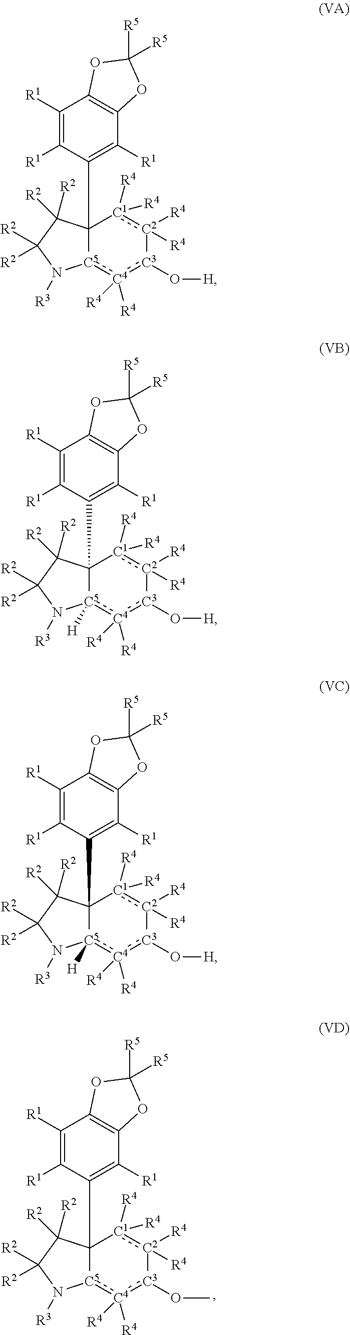 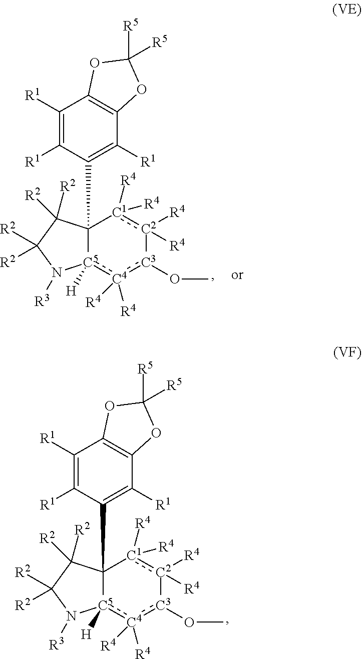
wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
A is O or S;
each R⁵ is independently H or a C₁-C₄ alkyl; and
where any one of the bonds between C¹ and C², C³ and C⁴, and/or C⁴ and C⁵ may be single bonds or double bonds.

2. The compound of claim 1, wherein at least one R is methyl.

3. The compound of claim 2, wherein R³ is methyl.

4. The compound of claim 3, wherein each R¹ and each R² is H.

5. The compound of claim 1, wherein each R⁵ is H.

6. The compound of claim 5, wherein R³ is methyl.

7. The compound of claim 1, wherein each R¹, each R², and each R⁴ is H.

8. The compound of claim 7, wherein R³ is methyl.

9. A pharmaceutical composition comprising one or more pharmaceutically acceptable carriers or excipients and
a compound having any one of any one of Formulas (VA), (VB), (VC), (VD), (VE), or (VF): 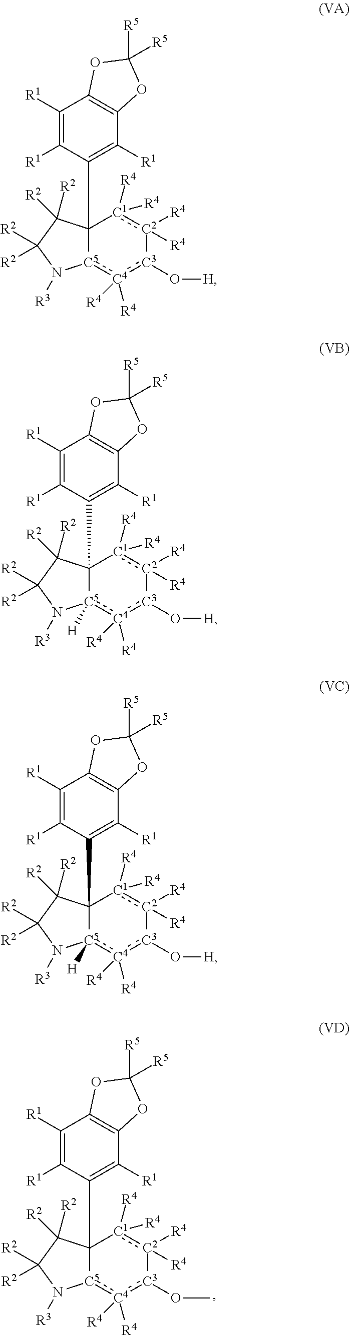 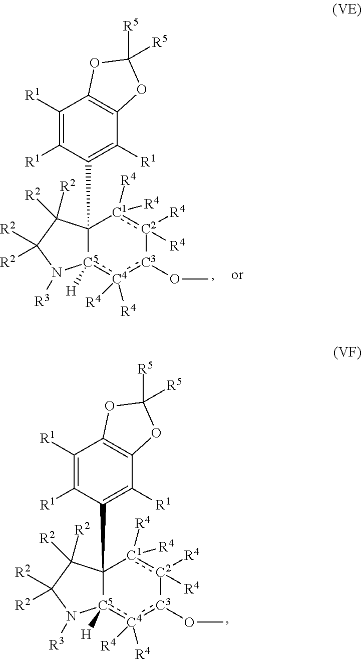
wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
A is O or S;
each R⁵ is independently H or a C₁-C₄ alkyl; and
where any one of the bonds between C¹ and C², C³ and C⁴, and/or C⁴ and C⁵ may be single bonds or double bonds.

10. A method of treating a subject having a depressive state, a psychological or psychiatric disorder with an anxiety component, alcohol and drug dependence, bulimia nervosa, PTSD, and/or obsessive-compulsive disorders comprising administering the pharmaceutical composition comprising one or more pharmaceutically acceptable carriers or excipients; and a compound having any one of any one of Formulas (VA), (VB), (VC), (VD), (VE), or (VF): 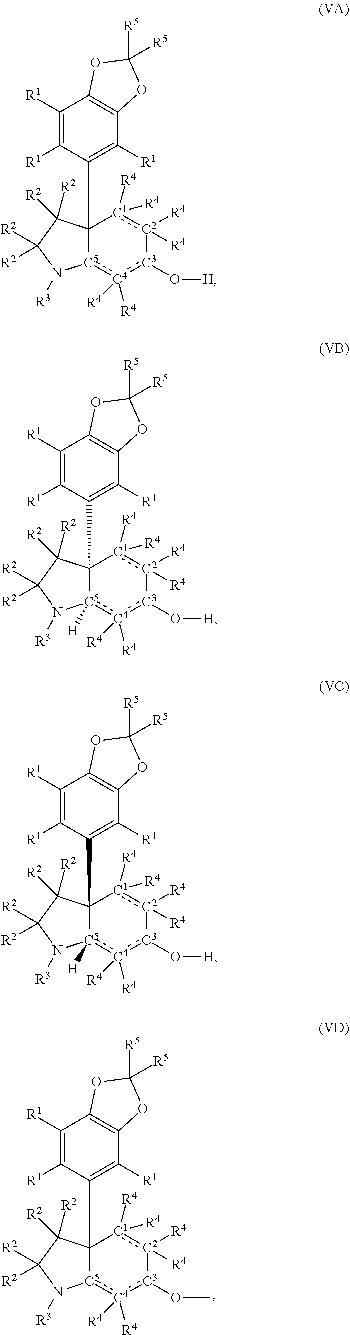 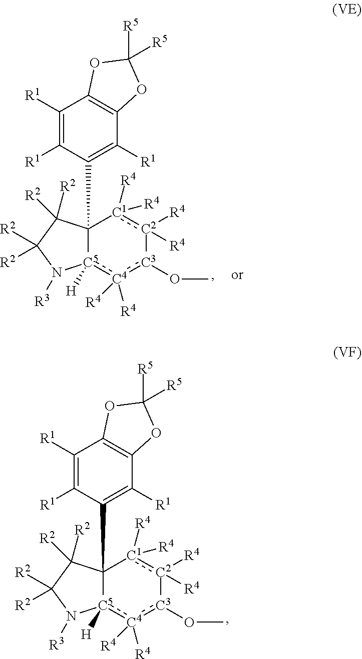
wherein
each R¹ is independently a substituted or unsubstituted C₁-C₄ alkyl group, a —O—C₁-C₄ alkyl group, or —OH;
each R² is independently a substituted or unsubstituted C₁-C₄ alkyl group;
R³ is —CH₃ or —CH₂—CH₃;
each R⁴ is independently a substituted or unsubstituted C₁-C₄ alkyl group;
A is O or S;
each R⁵ is independently H or a C₁-C₄ alkyl; and
where any one of the bonds between C¹ and C², C³ and C⁴, and/or C⁴ and C⁵ may be single bonds or double bonds.
